(19) United States
(12) Patent Application Publication
(10) Pub. No.: US 2011/0152700 A1
(43) Pub. Date: Jun. 23, 2011

(54) ELECTRONIC SPHYGMOMANOMETER FOR ENHANCING RELIABILITY OF MEASUREMENT VALUE

(75) Inventors: Yukiya Sawanoi, Nara-shi (JP); Hiroshi Kishimoto, Kyoto-shi (JP); Masaki Tomioka, Kyoto-shi (JP); Naomi Matsumura, Takatsuki-shi (JP); Reiji Fujita, Kyoto-shi (JP); Yuuichiro Tamaki, Kyoto-shi (JP); Shingo Yamashita, Kyoto-shi (JP)

(73) Assignee: OMRON HEALTHCARE CO., LTD., Kyoto (JP)

(21) Appl. No.: 13/041,044

(22) Filed: Mar. 4, 2011

Related U.S. Application Data
(63) Continuation of application No. PCT/JP2009/064426, filed on Aug. 18, 2009.

(30) Foreign Application Priority Data
Sep. 5, 2008 (JP) 2008-228627

(51) Int. Cl. A61B 5/0225

(52) U.S. Cl. 600/493

(57) ABSTRACT

An electronic sphygmomanometer has a cuff to be attached to a measurement site, a pressurization/depressurization unit for adjusting a pressure to be applied to the cuff, a pressure detection unit including a plurality of pressure sensors, for detecting cuff pressures in the cuff based on pressure information output from the plurality of pressure sensors, and a blood pressure calculating unit for calculating a blood pressure based on a change in the cuff pressures detected by the pressure detection unit. Blood pressure measurement and detection of abnormality on the plurality of pressure sensors are carried out based on the cuff pressures respectively corresponding to the plurality of pressure sensors detected according to the pieces of pressure information output from the plurality of pressure sensors.

### BACKGROUND OF THE INVENTION

1. Technical Field

The present invention relates to electronic sphygmomanometers, and in particular, to an electronic sphygmomanometer for enhancing the reliability of a blood pressure measurement value.

2. Background Art

The blood pressure is one of indices for analyzing the cardiovascular diseases. Performing the risk analysis of the cardiovascular disease based on the blood pressure is effective in preventing cardiovascular diseases such as apoplexy, cardiac arrest, and cardiac infarct. The early morning high blood pressure in which the blood pressure rises early in the morning is related to heart diseases, apoplexy, and the like. Furthermore, a symptom in which the blood pressure suddenly rises between one hour and one and a half hour after waking up, called as the morning surge, in the early morning high blood pressure is known to have a causal connection with apoplexy. Therefore, it is useful in the risk analysis of the cardiovascular diseases to grasp the mutual relationship between time (lifestyle habits) and a change in blood pressure. The blood pressure thus needs to be continuously measured over a long period of time.

From research outcomes of recent years, it is found that home blood pressures measured at home are more effective in preventing, diagnosing, and treating the cardiovascular diseases than the blood pressures (casual blood pressures) measured in hospitals and at the time of health checkups.

According to this finding, home sphygmomanometers are widely used, and the movements to use the home blood pressure values for diagnosis have been also starting.

According to Patent Document 1 (Japanese Unexamined Patent Publication No. 7-51233), the process for correcting an error of a measurement value dependent on the characteristics of a pressure sensor for the blood pressure measurement is carried out at the time of manufacture of an electronic sphygmomanometer in order to enhance the measurement accuracy of the sphygmomanometer.

Patent Document 1: Japanese Unexamined Patent Publication No. 7-51233

### SUMMARY

In Patent Document 1, the corrections related to the pressure sensors are carried out dependent on the differences in characteristics of the individual electronic sphygmomanometers at the time of manufacture of the electronic sphygmomanometers. Unlike the sphygmomanometers used in medical institutions such as hospitals, the home sphygmomanometer is generally not subjected to periodic calibration other than in a specific situation such as broken after the purchase thereof. Thus, even if the output of the pressure sensor, which is the most important in the blood pressure measurement, is shifted by greater than or equal to a defined allowable tolerance, there is no method of noticing such a phenomenon and it is unknown whether of not the blood pressure measurement value is correct. Thus, even if the blood pressure measurement value greatly differs from the normal blood pressure or the casual blood pressure, it is unknown whether the measurement value is really different from the blood pressure value or is different due to an error of the pressure sensor of the sphygmomanometer, which may cause a user to feel anxious.

In the sphygmomanometers for some medical institutions, two pressure sensors are mounted and the pressure is monitored based on the outputs of these pressure sensors. In such a sphygmomanometer, however, the functions of the two pressure sensors are used for different purposes. That is, the blood pressure is calculated with cuff pressure information obtained with one of the pressure sensors, and the abnormality detection is carried out based on the output of the other pressure sensor. Specifically, abnormality is detected when the detected pressure value of the pressure sensor greatly exceeds 300 mmHg, for example. In this case, a pump is stopped and a valve is opened to ensure safety. Therefore, the other pressure sensor is applied for safety countermeasures defined in the medical standard IEC 60601-2-30, and does not guarantee the accuracy of one of the pressure sensors used for the blood pressure measurement.

Therefore, the accuracy of the pressure sensor used for the blood pressure calculation needs to be guaranteed by this pressure sensor itself. To this end, an expensive pressure sensor needs to be used since required is a highly accurate pressure sensor that is not influenced by disturbance such as temperature changes and that has small change over the years. Furthermore, as the two pressure sensors having the functions for the different purposes are mounted, the failure rate of the sphygmomanometer due to the failures of the pressure sensors simply doubles compared to a sphygmomanometer with one pressure sensor.

According to one or more embodiments of the invention, an electronic sphygmomanometer includes: a cuff to be attached to a measurement site; a pressurization/depressurization unit for adjusting a pressure to be applied to the cuff; a pressure detection unit including a plurality of pressure sensors, for detecting cuff pressures in the cuff based on pressure information output from the plurality of pressure sensors; and a blood pressure calculating unit for calculating a blood pressure based on a change in the cuff pressures detected by the pressure detection unit, wherein blood pressure measurement and detection of abnormality on the plurality of pressure sensors are carried out based on the cuff pressures respectively corresponding to the plurality of pressure sensors detected according to the pieces of pressure information output from the plurality of pressure sensors.

According to one or more embodiments of the invention, the blood pressure calculating unit calculates the blood pressure based on the cuff pressures respectively corresponding to the plurality of pressure sensors.

The blood pressure calculating unit according to one or more embodiments of the invention calculates the blood pressure based on an average of the cuff pressures respectively corresponding to the plurality of pressure sensors.

The blood pressure calculating unit according to one or more embodiments of the invention calculates the blood pressure based on a median value of the cuff pressures respectively corresponding to the plurality of pressure sensors.

According to one or more embodiments of the invention, the electronic sphygmomanometer further includes an abnormality detecting portion for detecting abnormality of the plurality of pressure sensors. The abnormality detecting portion mutually compares the pieces of pressure information respectively corresponding to the plurality of pressure sensors and detects that at least one of the plurality of pressure sensors is abnormal based on a comparison result.

According to one or more embodiments of the invention, the blood pressure calculating unit excludes the cuff pressure corresponding to the pressure sensor that is detected as being abnormal, out of the plurality of pressure sensors from data used to calculate the blood pressure.

According to one or more embodiments of the invention, the electronic sphygmomanometer further includes a storage unit for storing data of the blood pressure calculated by the blood pressure calculating unit. A detection result by the abnormality detecting portion is stored in the storage unit in association with the data of the blood pressure.

According to one or more embodiments of the invention, the pressure information is detected in a process of increasing or in a process of decreasing the pressure to be applied to the cuff by the pressurization/depressurization unit.

According to one or more embodiments of the invention, the pressure information is detected in a pressurization process or in a depressurization process at each of the cuff pressures indicating a plurality of predetermined levels.

According to one or more embodiments of the invention, the abnormality detecting portion mutually compares the pieces of pressure information respectively corresponding to the plurality of pressure sensors and detects that at least one of the plurality of pressure sensors is abnormal based on the comparison result while the blood pressure measurement is not being carried out by the electronic sphygmomanometer.

The abnormality detecting portion according to one or more embodiments of the invention mutually compares the pieces of pressure information respectively corresponding to the plurality of pressure sensors and detects that at least one of the plurality of pressure sensors is abnormal based on the comparison result during a period from turning ON a power of the electronic sphygmomanometer to a measurable state.

According to one or more embodiments of the invention, the abnormality detecting portion mutually compares the pieces of pressure information respectively corresponding to the plurality of pressure sensors at a predetermined cuff pressure, and detects that at least one of the plurality of pressure sensors is abnormal based on a comparison result. The predetermined cuff pressure corresponding to each of the plurality of pressure sensors indicates a difference between pressure information output by the pressure sensor when a pressure of 0 mmHg is applied to the cuff in measuring the blood pressure and pressure information output by the pressure sensor when the pressure of 0 mmHg is applied to the cuff and acquired preliminarily in calibration of the pressure sensor.

According to one or more embodiments of the invention, the pieces of pressure information respectively corresponding to the plurality of pressure sensors are detected in a state where the cuff is wrapped around a member in a circular column shape and a predetermined amount of air is flowed into the cuff.

According to one or more embodiments of the invention, the electronic sphygmomanometer further includes a main body separate from the cuff. The main body includes the pressurization/depressurization unit, the pressure detection unit, the blood pressure calculating unit, and the abnormality detecting portion. A housing of the main body is configured by member in the circular column shape.

According to one or more embodiments of the invention, the electronic sphygmomanometer further includes: a tank for storing a predetermined amount of air; a first air flow path communicating to the pressurization/depressurization unit and to the pressure detection unit; a second air flow path communicating to the cuff; a third air flow path communicating to the tank; and a flow path switching unit for selectively connecting one of the second air flow path and the third air flow path to the first air flow path. When the predetermined amount of air is flowed into the tank after the flow path switching unit connects the third air flow path to the first air flow path, the pressure detection unit detects the cuff pressure in the cuff based on the pieces of pressure information output from the plurality of pressure sensors.

When reading out data of the blood pressure from the storage unit and outputting to outside, the abnormality detecting portion according to one or more embodiments of the invention mutually compares the pieces of pressure information respectively corresponding to the plurality of pressure sensors and detects that at least one of the plurality of pressure sensors is abnormal based on the comparison result.

According to one or more embodiments of the invention, the electronic sphygmomanometer further includes a timer for timing time. When setting the time of the timer, the abnormality detecting portion mutually compares the pieces of pressure information respectively corresponding to the plurality of pressure sensors and detects that at least one of the plurality of pressure sensors is abnormal based on the comparison result.

According to one or more embodiments of the invention, the electronic sphygmomanometer further includes a power supply unit including a battery. The abnormality detecting portion mutually compares the pieces of pressure information respectively corresponding to the plurality of pressure sensors and detects that at least one of the plurality of pressure sensors is abnormal based on the comparison result immediately after the battery of the power supply unit is changed to another battery.

According to one or more embodiments of the invention, the electronic sphygmomanometer further includes a power supply unit which is supplied with power from outside and which outputs the supplied power to each unit of the electronic sphygmomanometer. The abnormality detecting portion according to one or more embodiments of the invention mutually compares the pieces of pressure information respectively corresponding to the plurality of pressure sensors and detects that at least one of the plurality of pressure sensors is abnormal based on the comparison result immediately after supply of the power to the power supply unit from the outside is started.

According to one or more embodiments of the invention, the abnormality detecting portion mutually compares the pieces of pressure information respectively corresponding to the plurality of pressure sensors and detects that at least one of the plurality of pressure sensors is abnormal based on the comparison result according to an instruction input from outside.

The electronic sphygmomanometer according to one or more embodiments of the invention displays whether the plurality of pressure sensors are normal or abnormal based on the detection result of the abnormality detecting portion.

According to one or more embodiments of the invention, the electronic sphygmomanometer individually displays normality or abnormality of each of the plurality of pressure sensors based on the detection result of the abnormality detecting portion.

Whether the pressure sensor is normal or abnormal is indicated according to the cuff pressure detected in correspondence with the pressure sensor.

The electronic sphygmomanometer according to one or more embodiments of the invention displays whether the pressure sensor is normal or abnormal before the start of the blood pressure measurement.

The electronic sphygmomanometer according to one or more embodiments of the invention displays whether the pressure sensor is normal or abnormal at the time of displaying the blood pressure measurement result.

According to one or more embodiments of the invention, the electronic sphygmomanometer further includes a storage unit for storing data of the blood pressure calculated by the blood pressure calculating unit. The blood pressure measurement result includes the data of the blood pressure read out from the storage unit.

The electronic sphygmomanometer according to one or more embodiments of the invention displays whether the plurality of pressure sensors are normal or abnormal based on the detection result of the abnormality detecting portion every time the abnormality detecting portion detects that at least one of the plurality of pressure sensors is abnormal.

According to one or more embodiments of the present invention, the reliability of the blood pressure measurement value can be enhanced since the blood pressure measurement and the detection of abnormality on the plurality of pressure sensors can be carried out based on the cuff pressures detected using the plurality of pressure sensors.

### BRIEF DESCRIPTION OF THE DRAWINGS

FIG. 1 is an outer appearance view of an electronic sphygmomanometer according to one or more embodiments of the present invention.

FIG. 2 is a hardware configuration diagram of an electronic sphygmomanometer according to a first embodiment.

FIG. 3 is a function configuration diagram of the electronic sphygmomanometer according to the first embodiment.

FIG. 4 is a flowchart of a blood measurement process according to the first embodiment.

FIG. 5 is a view describing the procedure of calculating a blood pressure according to the first embodiment.

FIG. 6 is a graph indicating the characteristics of a pressure sensor.

FIG. 7 is a flowchart showing the procedure of canceling the blood pressure measurement operation according to the first embodiment.

FIG. 8 is a flowchart showing the processing procedure of not starting the blood pressure measurement operation according to the first embodiment.

FIG. 9 is a view showing the schematic appearance of an electronic sphygmomanometer according to a second embodiment.

FIG. 10 is a view showing the schematic appearance of the electronic sphygmomanometer according to the second embodiment.

FIG. 11 is a view showing the schematic appearance of another electronic sphygmomanometer according to the second embodiment.

FIG. 12 is a view showing the schematic appearance of still another electronic sphygmomanometer according to the second embodiment.

FIG. 13 is a flowchart of an abnormality detection process of the pressure sensor according to the second embodiment.

FIG. 14 is a hardware configuration diagram of an electronic sphygmomanometer according to a third embodiment.

FIG. 15 is a function configuration diagram of the electronic sphygmomanometer according to the third embodiment.

FIG. 16 is a flowchart of an abnormality detection process of the pressure sensor according to the third embodiment.

FIG. 17 is a view showing one example of a display according to one or more embodiments of the present invention.

FIG. 18 is a view showing another example of a display according to one or more embodiments of the present invention.

FIG. 19 is a view showing still another example of a display according to one or more embodiments of the present invention.

FIG. 20 is a view showing an outer appearance of a wrist type electronic sphygmomanometer.

### DETAILED DESCRIPTION

Embodiments of the present invention will be hereinafter described in detail with reference to the drawings. In each of the drawings, the same symbols refer to the same or corresponding portions, and the description thereof will not be repeated. In embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention.

### First Embodiment

An electronic sphygmomanometer mounted with two pressure sensors according to a first embodiment of the invention will be described. The measurement site is assumed to be an upper arm. The electronic sphygmomanometer calculates the blood pressure in accordance with the oscillometric method. The method applied to calculate the blood pressure is not limited to the oscillometric method.

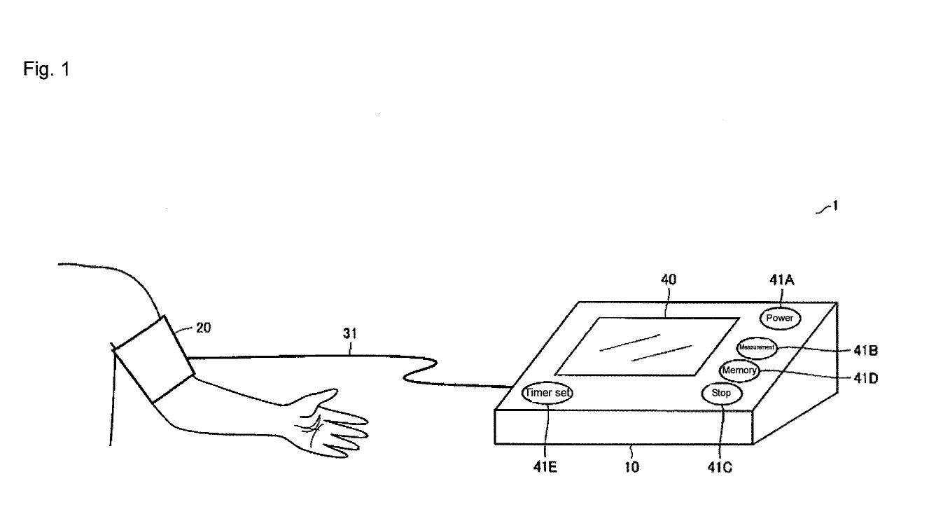

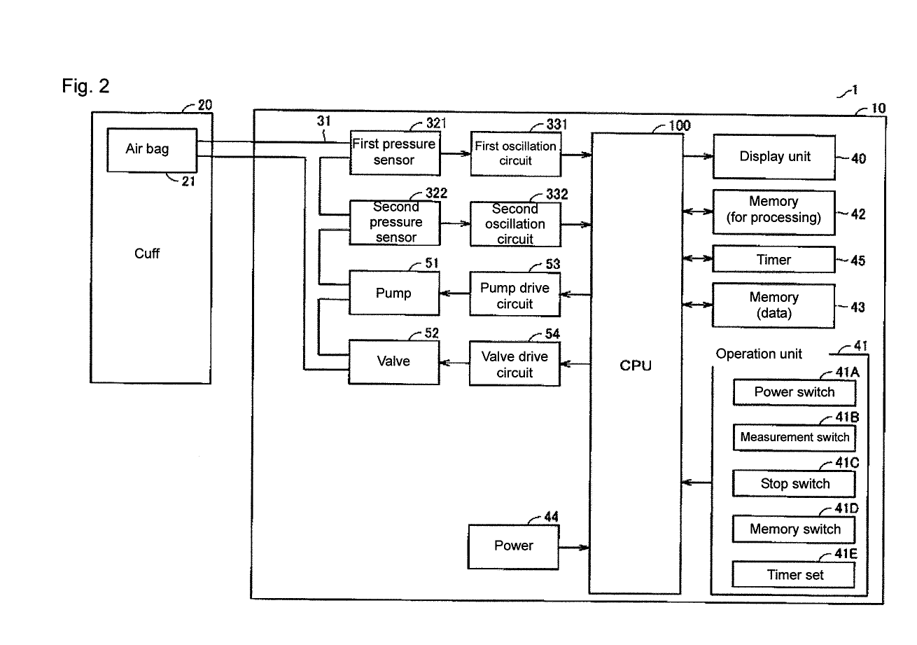

With reference to FIG. 1 and FIG. 2, an electronic sphygmomanometer 1 includes a main body 10, and a cuff 20 that can be wrapped around an upper arm of a person to be measured. The cuff 20 includes an air bag 21. A display unit 40 configured by liquid crystals or the like, and an operation unit 41 including a plurality of switches for accepting instructions from a user (person to be measured) are arranged on a surface of the main body 10.

The main body 10 includes the display unit 40 and the operation unit 41 described above. The main body 10 includes a CPU (Central Processing Unit) 100 for controlling the respective units in a concentrated manner and for carrying out various calculation processes, a memory 42 for processing for storing programs for causing the CPU 100 to perform predetermined operations and data, a memory 43 for storage for storing measured blood pressure data and the like, a power supply 44 for supplying power to the respective units of the main body 10, and a timer 45 for timing current time and outputting timing data to the CPU 100.

The operation unit 41 includes a power switch 41A for accepting an input of an instruction to turn ON or OFF the power supply, a measurement switch 41B for accepting an instruction to start measurement, a stop switch 41C for accepting an instruction to stop the measurement, a memory switch 41D for accepting an instruction to read out information such as the blood pressure data stored in the memory 43 from the memory 43 and displaying the same on the display unit 40, and a timer set switch 41E operated to set the timer 45.

The main body 10 further includes a mechanism for adjusting a cuff pressure, including a pump 51 and an exhaust valve (hereinafter referred to as valve) 52.

An air system including the pump 51, the valve 52, and first and second pressure sensors 321 and 322 for detecting pressures (cuff pressures) in the air bag 21 is connected to the air bag 21 included in the cuff 20 through an air tube 31.

The main body 10 also includes the above air system, the cuff pressure adjustment mechanism, and first and second oscillation circuits 331 and 332. The cuff pressure adjustment mechanism includes a pump drive circuit 53 and a valve drive circuit 54 as well as the pump 51 and the valve 52.

The pump 51 supplies air into the air bag 21 to increase the cuff pressure. The valve 52 is opened/closed to discharge or enclose the air in the air bag 21. The pump drive circuit 53 controls the drive of the pump 51 based on a control signal from the CPU 100. The valve drive circuit 54 controls the opening/closing of the valve 52 based on a control signal from the CPU 100.

The first and second pressure sensors 321 and 322 are capacitance sensors. The capacitance values of the first and second sensors 321 and 322 change according to the detected cuff pressures. The first and second oscillation circuits 331 and 322 are respectively connected to the corresponding pressure sensors and oscillate based on the capacitance values of the corresponding pressure sensors. The first and second oscillation circuits 331 and 332 each output a signal (hereinafter referred to as frequency signal) having a frequency corresponding to the capacitance value of the corresponding pressure sensor. The frequency signals output by the first and second oscillation circuits 331 and 332 are provided to the CPU 100. The CPU 100 converts the frequency signal input from the first oscillation circuit 331 or the second oscillation circuit 332 to a pressure to detect the pressure.

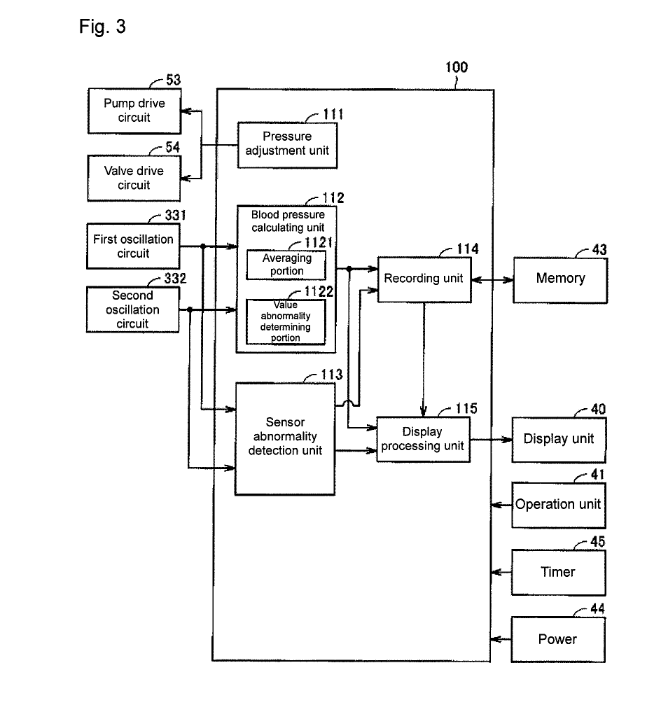

FIG. 3 shows the function configuration of the electronic sphygmomanometer 1. The CPU 100 includes a pressure adjustment unit 111, a blood pressure calculating unit 112, a sensor abnormality detection unit 113, a recording unit 114, and a display processing unit 115.

The pressure adjustment unit 111 controls the pump 51 and the valve 52 through the pump drive circuit 53 and the valve drive circuit 54, and also adjusts the cuff pressure by flowing/discharging the air into/from the air bag 21 through the air tube 31.

The blood pressure calculating unit 112 detects pulse wave amplitude information based on the frequency signal (this frequency signal indicates a pressure information signal) input from the first oscillation circuit 331 or the second oscillation circuit 332. A systolic blood pressure and a diastolic blood pressure are calculated according to the oscillometric method based on the detected pulse wave amplitude information, and a pulse rate per predetermined time is also calculated based on the detected pulse wave amplitude information. Specifically, the pulse wave amplitude information is detected based on the cuff pressure input from the first oscillation circuit 331 or the second oscillation circuit 332 in the process of gradually increasing (or decreasing) the cuff pressure to a predetermined value by means of the pressure adjustment unit 111, and the systolic blood pressure and the diastolic blood pressure of the person to be measured are calculated based on the detected pulse wave amplitude information. A conventionally known method can be applied to the calculation of the blood pressure and the calculation of the pulse rate according to the oscillometric method by the blood pressure calculating unit 112.

The sensor abnormality detection unit 113 receives the frequency signals output from the first oscillation circuit 331 and the second oscillation circuit 332 and analyzes the received signals to detect the abnormality of the first pressure sensor 321 and the second pressure sensor 322.

The recording unit 114 has a function of reading out data from the memory 43 or writing data to the memory 43. Specifically, the recording unit 114 receives data output from the blood pressure calculating unit 112, and stores the received data (blood pressure measurement data) in a predetermined storage region of the memory 43. Furthermore, the recording unit 114 receives data output from the sensor abnormality detection unit 113, and stores the received data (detection result of abnormality of pressure sensor) in a predetermined storage region of the memory 43. The recording unit 114 also reads out the measurement data from the predetermined storage region of the memory 43 based on the operation of the memory switch 41D of the operation unit 41, and provides the read measurement data to the display processing unit 115.

The display processing unit 115 receives the provided data and converts the input data into a displayable format to display the same on the display unit 40.

In FIG. 3, only the portion to directly perform input and output operations with the CPU 100 is shown in the peripheral circuit of the CPU 100.

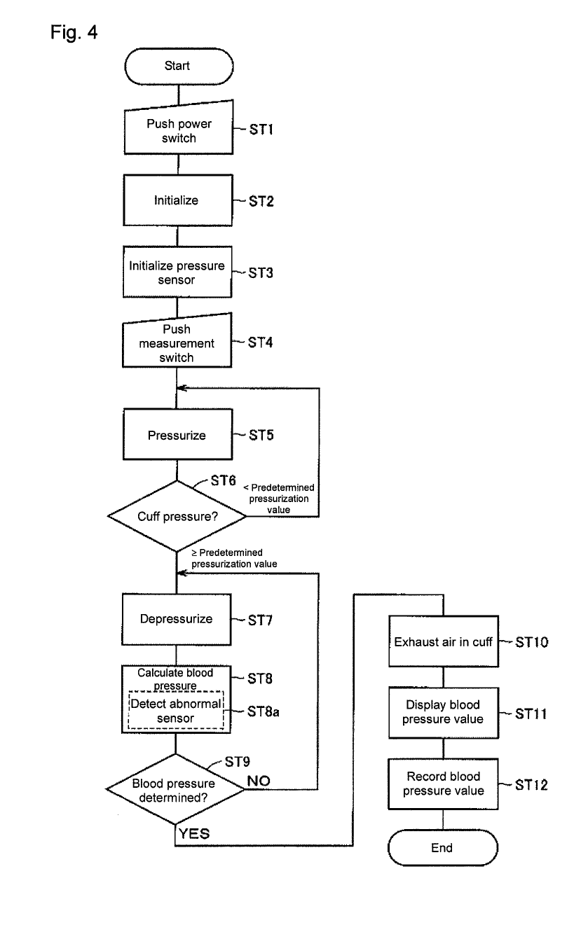

The processing procedure of the blood pressure measurement will be described with reference to FIG. 4. The flowchart of FIG. 4 is stored preliminarily in the memory 42 as a program. The blood pressure measurement process shown in FIG. 4 is realized when the CPU 100 reads out the program from the memory 42 and executes the command of the program thus read out.

First, when the person to be measured operates (pushes) the power switch 41A (step ST1), the CPU 100 initializes a work memory (not shown) (ST2).

The adjustments to 0 mmHg of the first and second pressure sensors 321 and 322 are then performed (ST3).

The person to be measured then attaches the cuff 20 by wrapping the same around the measurement site as shown in FIG. 1. After wrapping the cuff 20 around, the person to be measured operates (pushes) the measurement switch 41B (step ST4), so that the pressure adjustment unit 111 outputs control signals to the pump drive circuit 53 and the valve drive circuit 54. The pump drive circuit 53 and the valve drive circuit 54 close the valve 52 and then drive the pump 51 based on the control signals. The cuff pressure is thereby gradually increased to a predetermined pressure (steps ST5, ST6).

When the condition (cuff pressure predetermined pressurization value) is established in step ST6 after being pressurized to the predetermined pressure, the pressure adjustment unit 111 outputs the control signals to the pump drive circuit 53 and the valve drive circuit 54. The pump drive circuit 53 and the valve drive circuit 54 control to stop the pump 51 and then gradually open the valve 52 based on the control signals. The cuff pressure is thereby gradually decreased (step ST7).

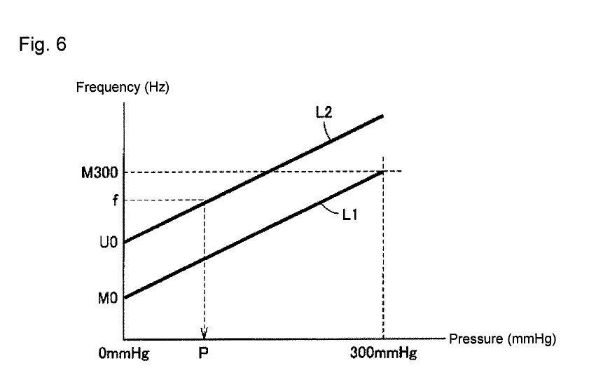

In the depressurization process, the blood pressure calculating unit 112 detects the pulse wave amplitude information based on the frequency signal output from the first oscillation circuit 331 or the second oscillation circuit 332, that is, based on a cuff pressure signal detected by the first pressure sensor 321 or the second pressure sensor 322, and performs a predetermined calculation using the detected pulse wave amplitude information. The systolic blood pressure and the diastolic blood pressure are calculated by this calculation (steps ST8, ST9). The pulse wave amplitude information indicates a volume change component of an artery of the measurement site, and is contained in the cuff pressure signal to be detected. The calculation according to the change in the characteristics of the pressure sensor is performed in the calculation of the blood pressure by the blood pressure calculating unit 112, which will be described later in FIG. 6. The blood pressure measurement is not limited to be carried out in the depressurization process, but may be carried out in the pressurization process (step ST5).

After the systolic blood pressure and the diastolic blood pressure are calculated and determined (YES in step ST9), the pressure adjustment unit 111 fully opens the valve 52 through the valve drive circuit 54. The air in the cuff 20 is thereby rapidly exhausted (step ST10).

The data of the blood pressure calculated by the blood pressure calculating unit 112 is provided to the display processing unit 115 and the recording unit 114. The display processing unit 115 receives the provided blood pressure data, and displays the received blood pressure data on the display unit 40 (step ST11). The recording unit 114 receives the provided blood pressure data, and stores the received blood pressure data in a predetermined storage region of the memory 43 in association with time data received from the timer 45 (step ST12).

The blood pressure calculating unit 112 can also calculate the pulse rate based on the detected pulse wave amplitude information. The calculated pulse rate is displayed on the display unit 40 by the display processing unit 115, and is also stored in the memory 43 in association with the blood pressure data by the recording unit 114.

The operations described so far are similar to those of a conventional sphygmomanometer.

In the conventional sphygmomanometer, the user cannot determine whether the pressure sensor is normal or abnormal, which is the most important element in calculating a blood pressure. Thus, in a case where the blood pressure measurement value greatly differs (e.g., different by 10 mmHg or more) from a normal value (e.g., measurement value of the previous day, measurement value in a hospital, or the like), the person to be measured may not know whether such a difference is caused by the physiological information of the living body or by a failure of the pressure sensor, and thus may have a sense of concern.

In this regard, the electronic sphygmomanometer 1 calculates an average value of the cuff pressures detected by the two pressure sensors 321 and 322 as a blood pressure. Therefore, even if the detection accuracy of one of the pressure sensors varies due to the change over the years, the reliability of the blood pressure measurement value can be enhanced by calculating the average value.

### (Calculation of Average Value)

At the time of calculating the blood pressure (step ST8 of FIG. 4), an averaging portion 1121 of the blood pressure calculating unit 112 receives the cuff pressures detected by the first and second pressure sensors 321 and 322 through the first and second oscillation circuits 331 and 332. An output signal of the first pressure sensor 321 is referred to as a cuff pressure a, and an output signal of the second pressure sensor 322 is referred to as a cuff pressure b. The averaging portion 1121 extracts the pulse wave amplitude information based on frequency signals corresponding to the cuff pressures a and b received from the first and second oscillation circuits 331 and 332. The blood pressure value is then calculated based on the extracted pulse wave amplitude information. In other words, a systolic blood pressure SBPa and a diastolic blood pressure DBPa are calculated based on the pulse wave amplitude information of the cuff pressure a, and a systolic blood pressure SBPb and a diastolic blood pressure DBPb are calculated based on the pulse wave amplitude information of the cuff pressure b. The average value of the systolic blood pressures SBPa and SBPb, and the average value of the diastolic blood pressures DBPa and DBPb are then calculated. The calculated average blood pressure values are displayed on the display unit 40 and stored in the memory 43. The blood pressure measurement value having high reliability is thereby obtained.

When using three or more pressure sensors, a median value may be used instead of the average value. If there is a pressure sensor that outputs a value having a large difference from the output value of another pressure sensor in the pressure sensors, the output value of this pressure sensor may be excluded in the calculation of the average value or the median value.

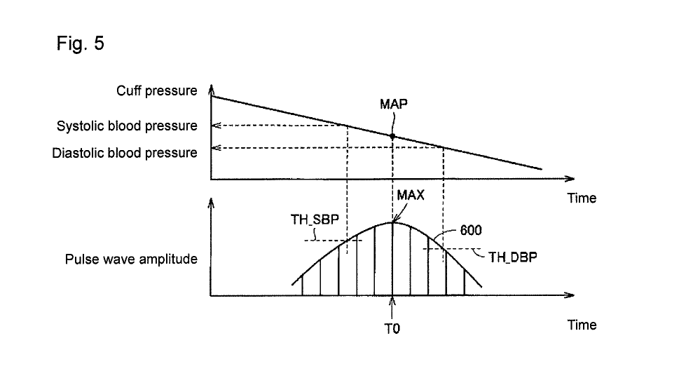

A concept of the blood pressure calculating method by the oscillometric method according to the first embodiment will be described below. The cuff pressure to be gradually decreased is indicated along a time axis timed by the timer 45 at the upper level of FIG. 5, and an envelope curve 600 of the pulse wave amplitude corresponding to the above pulse wave amplitude information is indicated along the same time axis at the lower level. The envelope curve 600 of the pulse wave amplitude is detected by extracting a pulse wave amplitude signal superimposed on the signal (cuff pressure) output from the pressure sensor in time series.

With reference to FIG. 5, after detecting a maximum value MAX of the amplitude on the envelope curve of the pulse wave amplitude, the blood pressure calculating unit 112 calculates two threshold values TH_DBP and TH_SBP by multiplying by predetermined constants (e.g., 0.7 and 0.5) the detected maximum value MAX. The blood pressure calculating unit 112 calculates as the diastolic blood pressure a cuff pressure at a point where the threshold value TH_DBP and the envelope curve 600 intersect on a side in which the cuff pressure is lower than a cuff pressure MAP (average blood pressure) detected at a time point T0 when the maximum value MAX is detected. The blood pressure calculating unit 112 also calculates s the systolic blood pressure a cuff pressure at a point where the threshold value TH_SBP and the envelope curve 600 intersect a on a side in which the cuff pressure is higher than the cuff pressure MAP.

Alternatively, the following calculation method may be adopted. Specifically, at the time of calculating the blood pressure (step ST8 of FIG. 4), the averaging portion 1121 receives the signals of the cuff pressures a and b from the first and second oscillation circuits 331 and 332. The pulse wave amplitude information is detected based on the average value of the signals of the received cuff pressures a and b, and the systolic blood pressure SBP and the diastolic blood pressure DBP are calculated based on the detected pulse wave amplitude information.

### (Determination of Sensor Abnormality)

In order to enhance the reliability of the blood pressure measurement value, at the time of calculating the blood pressure (step ST8 of FIG. 4), a value abnormality determining portion 1122 of the blood pressure calculating unit 112 receives the systolic blood pressures SBPa and SBPb, and the diastolic blood pressures DBPa and DBPb respectively calculated. The input systolic blood pressures SBPa and SBPb are compared with each other to detect a difference between the two pressures. Similarly, a difference is detected between the diastolic blood pressures DBPa and DBPb. The detected differences are then compared with a predetermined value (e.g., 5 mmHg), respectively. If determined that one or both of the differences exceed the predetermined value based on the comparison result, one of the pressure sensors is determined as abnormal.

Alternatively, the value abnormality determining portion 1122 compares the cuff pressures a and b indicated by the frequency singles input from the first and second oscillation circuits 331 and 332, and determines that one of the pressure sensors is abnormal if determined that the difference exceeds a predetermined value (e.g., 5 mmHg) based on the comparison result.

If determined that one of the pressure sensors is abnormal by the value abnormality determining portion 1122, the blood pressure calculating unit 112 does not use to display or record, that is, discards the calculated blood pressure measurement data based on the determination result. The reliability of the blood pressure measurement value thus can be enhanced. The blood pressure measurement data may be displayed on the display unit 40 along with information (message) indicating that the pressure sensor is abnormal, instead of being discarded. The blood pressure measurement data may be stored in the memory 43 in association with a flag indicating that the pressure sensor is abnormal.

### (Blood Pressure Calculation According to Characteristic Change of Pressure Sensor)

The pressure sensor is normally subjected to calibration at the time of manufacture of the electronic sphygmomanometer 1. At the time of calibration, an output (indicating the frequency of the output signal of the oscillation circuit in the first embodiment) in a case where the pressure sensor detects a predetermined pressure value (0 mmHg, 300 mmHg) is measured, and the measured value is stored in a predetermined storage region of the memory 43. The predetermined pressure value (0 mmHg, 300 mmHg) relies on being designed such that the blood pressure of 0 to 299 mmHg can be measured with the electronic sphygmomanometer 1. The measurement value is not rewritable in the memory 43, and cannot be erased. In the first embodiment as well, the value measured when calibration is performed in the manufacture of the first and second pressure sensors 321 and 322 is stored in a predetermined storage region of the memory 43.

At the time of the blood pressure measurement, the measurement values as the output of the first and second pressure sensors 321 and 322 to be input through the first and second oscillation circuits 331 and 332 are also stored in the memory 43 in the initialization of the pressure sensors (step ST3 of FIG. 4). When measuring the blood pressure, the blood pressure calculating unit 112 compares the measurement values of the calibrated outputs of the pressure sensors at the time of manufacture read from the memory 43 and the measurement values of the outputs of the pressure sensors in the initialization, and performs the 0 mmHg correction of the pressure sensors at the current time point based on the comparison result.

Specifically, assuming that the measurement values of the outputs of the pressure sensors in a case of being calibrated for 0 mmHg and 300 mmHg at the time of manufacture as M0 and M300, respectively, the measurement values of the outputs in the initialization of the pressure sensors as U0, and the frequency of the signal currently output from the oscillation circuit is ‘f’, the blood pressure calculating unit 112 calculates a pressure value P (mmHg) according to (Equation 1). The calculated pressure value P corresponds to the cuff pressure indicated in the upper level of FIG. 5.

Pressure value P={(f−U0−M0)÷(M300−M0)}×300 (Equation 1)

The calculation of the pressure value P according to (Equation 1) above will be further described with reference to the graph on the characteristics of the pressure sensor in FIG. 6. In the graph of FIG. 6, a pressure (mmHg) as the cuff pressure is indicated on the horizontal axis, and a frequency (Hz) of an output signal of an oscillation circuit is indicated on the vertical axis. In FIG. 6, characteristics L1 of the pressure sensor at the time of manufacture of the electronic sphygmomanometer 1 and current characteristics L2 of the pressure sensor are indicated.

If the characteristics L1 of the pressure sensor at the time of manufacture and the current characteristics L2 of the pressure sensor are the same, Equation (2) below is satisfied.

Pressure value P=(f−M0)/(M300−M0)×300 (Equation 2)

Actually, the characteristics of the pressure sensor cannot maintain the characteristics L1 at the time of manufacture due to various factors such as usage situations, and for example, the characteristics L1 change to the current characteristics L2. Therefore, (Equation 2) is replaced by (Equation 1) using the output U0 in the initialization of the pressure sensor generated with the change of the characteristics.

### (Abnormality Detection of Sensor)

The abnormality detection of the pressure sensor is performed by the sensor abnormality detection unit 113 (step ST8a) in parallel to the process (step ST8) by the blood pressure calculating unit 112 according to (Equation 1) above.

The sensor abnormality detection unit 113 reads the outputs U0 and M0 corresponding to the first and second pressure sensors 321 and 322 from the memory 43. A difference between the read outputs M0 and U0 is calculated on each of the pressure sensors, and the calculated difference and a predetermined value are compared with each other. If detected that the difference exceeds the predetermined value as a result, the pressure sensor corresponding to the outputs U0 and M0, from which the difference is calculated, is determined as abnormal.

If the difference (difference between the outputs M0 and U0) is smaller than or equal to the predetermined value but there is a difference in the pressure values indicated by the output signals of the pressure sensors in both of the first and second pressure sensors 321 and 322, a determination is made such that one of the pressure sensors is abnormal. Specifically, the pressure sensor with a larger calculated difference (difference between the outputs M0 and U0) out of the first and second pressure sensors 321 and 322 is specified as abnormal.

The detection result of the sensor abnormality detection unit 113 may be displayed on the display unit 40 through the display processing unit 115. The person to be measured who checked the display then can know whether or not the pressure sensor is abnormal, and hence can obtain a certain sense of security even if the blood pressure measurement result is deviated from the normal value. The insecurity to the accuracy of the blood pressure measurement value can also be removed. The detection result of the sensor abnormality detection unit 113 may be stored in the memory 43 in association with the blood pressure measurement value through the recording unit 114.

Therefore, even if one of the two pressure sensors is detected as abnormal, the blood pressure can be calculated using the other pressure sensor, and thus the failure rate of the electronic sphygmomanometer 1 caused by the pressure sensors can be reduced to ½.

### (Process of Canceling Blood Pressure Measurement)

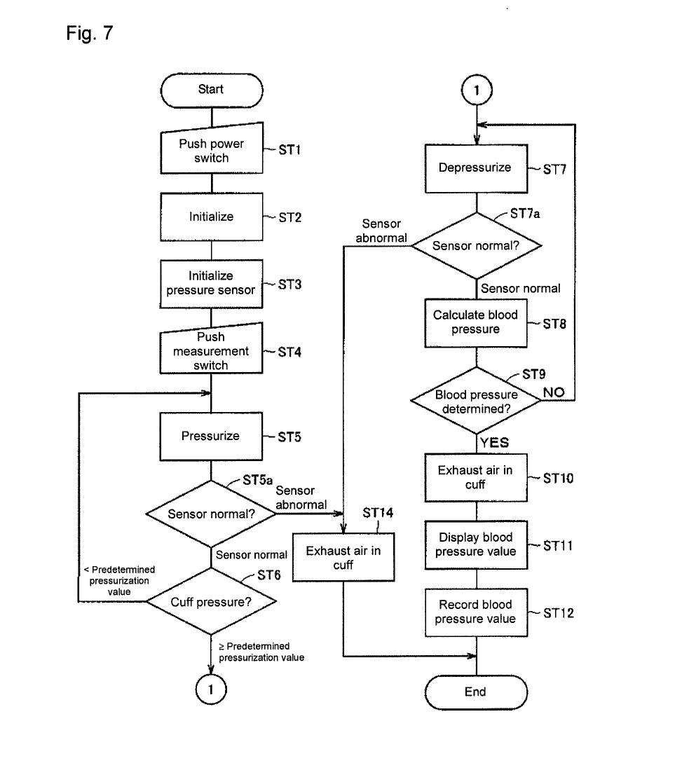

The procedure of canceling the blood pressure measurement in a case where abnormality of a pressure sensor is detected in the pressurization process or the depressurization process will be described with reference to the flowchart in FIG. 7.

FIG. 7 shows the flowchart in which the processes in steps ST5a, ST7a, and ST14 are added to the processes of the blood pressure measurement included in FIG. 4. The remaining processes in FIG. 7 are the same as those described in FIG. 4, and thus the added processes will be described herein.

In the pressurization process (step ST5) or the depressurization process (step ST7), whether the first and second pressure sensors 321 and 322 are normally operating is detected according to the procedure of (abnormality detection of sensor) by the sensor abnormality detection unit 113 (steps ST5a, ST7a). If detected as normally operating (“sensor normal” in step ST5a or 7a), the blood pressure measurement is performed by continuing the pressurizing operation and the depressurizing operation.

On the other hand, if detected that the pressure sensor is abnormal in step ST5a or ST7a (“sensor abnormal” in step ST5a or 7a), the process proceeds to step ST14, and the operation for canceling the blood pressure measurement is executed. Specifically, the air in the air bag 21 of the cuff 20 is rapidly exhausted. Thereafter, the blood pressure measurement is terminated. In this case, a message indicating that “the blood pressure measurement is canceled due to abnormality of the pressure sensor” may be output to the display unit 40 in order to notify the person to be measured of the reason for canceling the blood pressure measurement. The rapid exhaust is realized when the pressure adjustment unit 111 fully opens the valve 52 through the valve drive circuit 54.

The pressurization process or the depressurization process may be carried out every time the cuff pressure to be detected reaches a plurality of predetermined cuff pressures.

### (Process of Not Starting Blood Pressure Measurement at the Time of Sensor Abnormality)

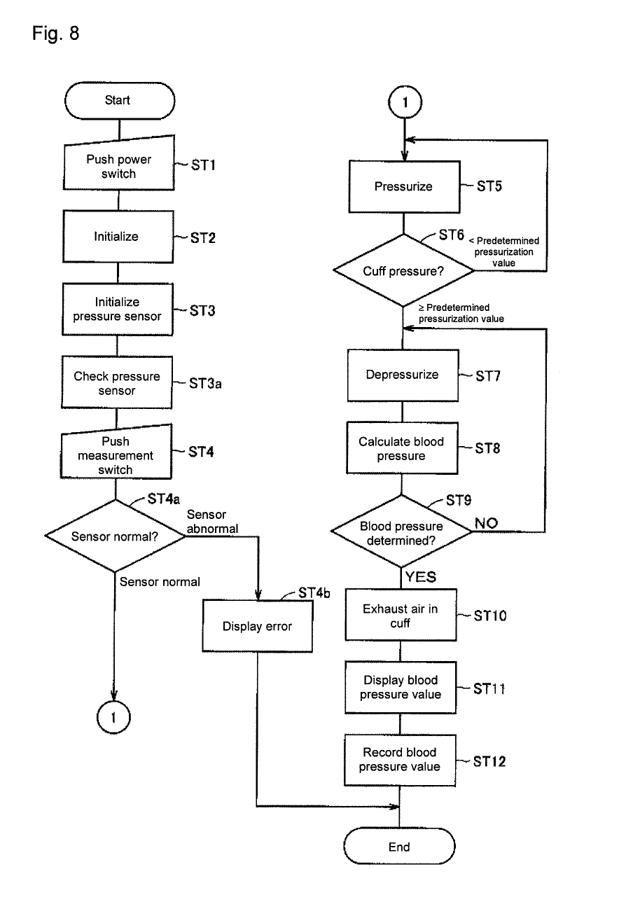

The procedure of not starting the blood pressure measurement when the abnormality of the pressure sensor is detected even if the measurement switch 41B is operated will now be described with reference to the flowchart in FIG. 8.

FIG. 8 shows the flowchart in which the processes in steps ST3a, ST4a, and ST4b are added to the processes of the blood pressure measurement in FIG. 4. The remaining processes in FIG. 8 are the same as those described in FIG. 4, and thus the added processes will be described herein.

With reference to FIG. 8, after the processes in steps ST1 to ST3 are executed similarly to the above description, whether or not the first and second pressure sensors 321 and 322 are normally operating is detected according to the procedure of (abnormality detection of sensor) by the sensor abnormality detection unit 113 (steps ST3a).

After the measurement switch 41B is operated by the person to be measured and the start of the blood pressure measurement is instructed (step ST4), the process proceeds to step ST5 if determined that the pressure sensor is normally operating (“sensor normal” in step ST4a) based on the detection result in step ST3a, and the subsequent blood pressure measurement process is started.

On the other hand, if determined that the pressure sensor is abnormally operating (“sensor abnormal” in step ST4a) based on the detection result in step ST3a, the process of displaying an error is performed (step ST4b). More specifically, the sensor abnormality detection unit 113 provides a signal indicating that the abnormality of the sensor is detected to the display processing unit 115, and hence the display processing unit 115 displays an error message notifying that the sensor abnormality has occurred on the display unit 40 based on the provided signal. The person to be measured can know the reason why the blood pressure measurement cannot be started by checking this message. The blood pressure measurement process is thereafter terminated.

### Second Embodiment

According to a second embodiment of the present invention, an electronic sphygmomanometer 1A has an outer appearance different from the electronic sphygmomanometer 1 of FIG. 1 applied in the first embodiment. The function configuration of FIG. 2 is realized also in the electronic sphygmomanometer 1A. Furthermore, the procedure of abnormality detection of the pressure sensor and the procedure of the blood pressure measurement described in the first embodiment can also be applied to the electronic sphygmomanometer 1A.

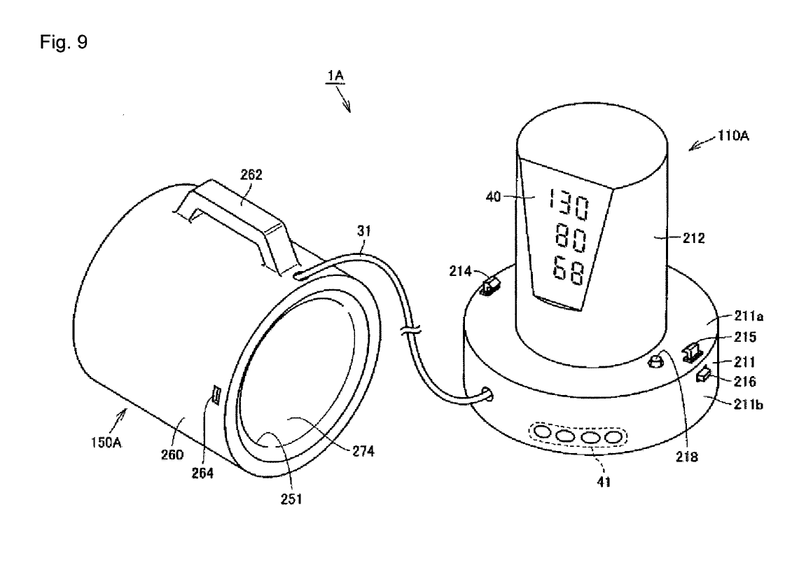

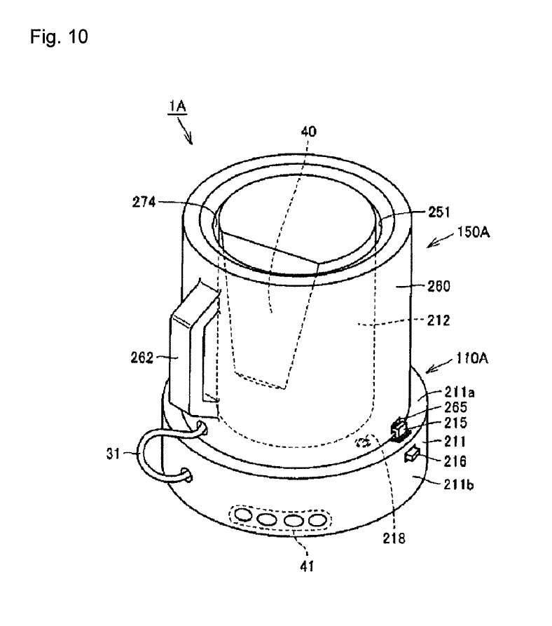

FIG. 9 and FIG. 10 each show the outer appearance of the electronic sphygmomanometer 1A. The schematic appearance of the electronic sphygmomanometer 1A in a state where a cuff is detached from a main body is shown in FIG. 9, and the schematic appearance of the electronic sphygmomanometer 1A in a state where the cuff is attached to the main body is shown in FIG. 10.

As shown in FIG. 9 and FIG. 10, the electronic sphygmomanometer 1A mainly includes a main body 110A and a cuff 150A. The main body 110A and the cuff 150A are connected with each other by an air tube 31 that serves as an air path. The air tube 31 is provided as a tubular member having an appropriate degree of flexibility.

The main body 110A includes a base portion 211 to be mounted on a mounting board such as a table, and an accommodated portion 212. The accommodated portion 212 corresponds to a portion projecting upward from an upper surface 211a of the base portion 211 in a state where the main body 110A is mounted on the board. The accommodated portion 212 is a site to be covered by the cuff 150A of the electronic sphygmomanometer 1A not in use. The accommodated portion 212 is formed in a substantially circular column shape with a resin material having rigidity. The electronic sphygmomanometer 1A has the display unit 40 on a peripheral surface of the accommodated portion 212, and the operation unit 41 on a peripheral surface 211b on a side of the base portion 211.

The electronic sphygmomanometer 1A includes a microswitch 218 serving as a detection unit for detecting whether or not the cuff 150A is set to the main body 110A at a predetermined position on the upper surface 211a of the base portion 211. The electronic sphygmomanometer 1A includes a fixing hook 214 and serving as a fixing portion for fixing the cuff 150A to the main body 110A when the cuff 150A is set to the main body 110A, and a movable hook 215. A release button 216 is arranged on the peripheral surface 211b of the base portion 211. The release button 216 is provided in association with the movable hook 215. The person to be measured operates the release button 216 to release the engagement of the cuff 150A by the fixing hook 214 and the movable hook 215.

The cuff 150A has an outer shape formed in a substantially cylindrical shape so as to be attachable to an upper arm as the measurement site of the person to be measured when using the electronic sphygmomanometer 1A. The cuff 150A includes the air bag 21 serving as a fluid bag for compressing the upper arm, a shell 260 as a substantially cylindrical frame formed to cover the outer side of the air bag 21, and a cuff cover 274 for covering the inner side of the air bag 21. The air bag 21 is arranged along an inner peripheral surface of the shell 260, and as a result, the cuff 150A has a hollow portion 251 to which the upper arm can be inserted when in use.

A handle 262 that can be gripped with the other hand of the upper arm to which the cuff 150A is attached is arranged at a predetermined position on the peripheral surface of the shell 260 so as to facilitate the attachment/detachment task of the cuff 150A to/from the upper arm. Recesses 264, 265 that engage the fixing hook 214 and the movable hook 216 described above are provided at predetermined positions on the peripheral surface of the shell 260.

The detection operation by the microswitch 218 will be described. The microswitch 218 is arranged on the upper surface 211a of the base portion 211 of the main body 110A. The microswitch 218 is arranged to be positioned to project upward from the upper surface 211a of the base portion 211 when a switch portion thereof is not pushed. The switch portion of the microswitch 218 is pushed down in the downward direction in the figure by an end surface face in the axial direction of the cuff 150A when the cuff 150A is attached to the main body 110A. Thus, whether or not the cuff 150A is attached to the main body 110A, that is, whether or not the accommodated portion 212 is accommodated in the cuff 150A, is detected by the microswitch 218.

As shown in FIG. 9, in the electronic sphygmomanometer 1A, the upper arm can be inserted in the axial direction to the hollow portion 251 formed in the cuff 150A when the cuff 150A is detached from the main body 110A so as to be brought into an usage state where the cuff 150A can be attached to the upper arm. On the other hand, as shown in FIG. 10, the accommodated portion 212 of the main body 110A is accommodated in the hollow portion 251 of the cuff 150A when the cuff 150A is attached to the main body 110A so as to be brought into a non-usage state where the electronic sphygmomanometer 1A is not in use. In the non-usage state, the display unit 40 and the operation unit 41 arranged to the main body 110A are covered by the cuff 150A.

### (Other Configurations)

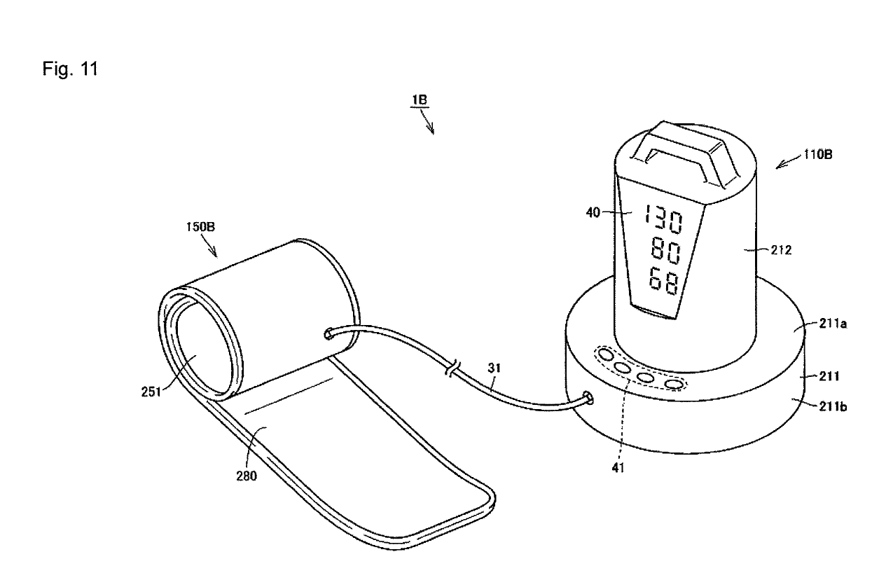

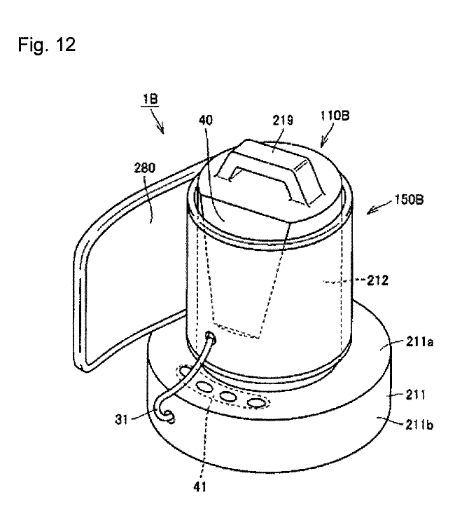

The configuration of attaching the cuff to the main body in the substantially circular column shape in the electronic sphygmomanometer not in use may be such a configuration shown in FIG. 11 or FIG. 12.

FIG. 11 shows a state where the cuff is detached from the main body, and FIG. 12 shows a state where the cuff is attached to the main body. In FIG. 11 and FIG. 12, the same symbols are denoted in the figures for the portions similar to those of the electronic sphygmomanometer 1A, and the description thereof will not be repeated again.

Similarly to the electronic sphygmomanometer 1A, an electronic sphygmomanometer 1B shown in FIG. 11 and FIG. 12 mainly includes a main body 110B and a cuff 150B. The main body 110B and the cuff 150B are connected with each other by the air tube 31. The configuration of the main body 150B is similar to that of the main body 110A except that the mechanism for fixing the cuff 150B to the main body 110B in the non-usage state where the cuff 150B is attached to the main body 110B is not arranged, and that the detection mechanism for detecting whether or not the cuff 150B is attached to the main body 110B is not arranged.

The cuff 150B has an outer shape of the site to be attached to the upper arm of the person to be measured when using the electronic sphygmomanometer 1B in a substantially cylindrical shape. The cuff 150B includes the air bag 21 serving as the fluid bag for compressing the upper arm, a curler (not shown), and a cover body 280 serving as a bag-shaped exterior member for including the air bag 21 and the curler. The curler is a curved elastic plate that is arranged on the outer side of the air bag 21 and biases the air bag 21 toward the upper arm when the cuff 150B is wrapped around the upper arm. The cuff 150B includes the hollow portion 251 to which the upper arm can be inserted in the usage state.

The electronic sphygmomanometer 1B is configured to be brought into two states, namely, a state where the cuff 150B is attached to the main body 110B and a state where the cuff 150B is detached from the main body 110B. As shown in FIG. 11, when the cuff 150B is detached from the main body 110B, there is achieved the usage state where the cuff 150B can be attached to the upper arm by inserting the upper arm to the hollow portion 251 formed in the cuff 150B. On the other hand, as shown in FIG. 12, when the cuff 150B is attached to the main body 110B, the accommodated portion 212 of the main body 110B is accommodated in the hollow portion 251 of the cuff 150B, and there is achieved the non-usage state where the electronic sphygmomanometer 1B is not used. As shown in FIG. 12, in the non-usage state, the display unit 40 arranged to the main body 110B is covered by the cuff 150B.

### (Another Example of Abnormality Detection in Initialization of Pressure Sensor)

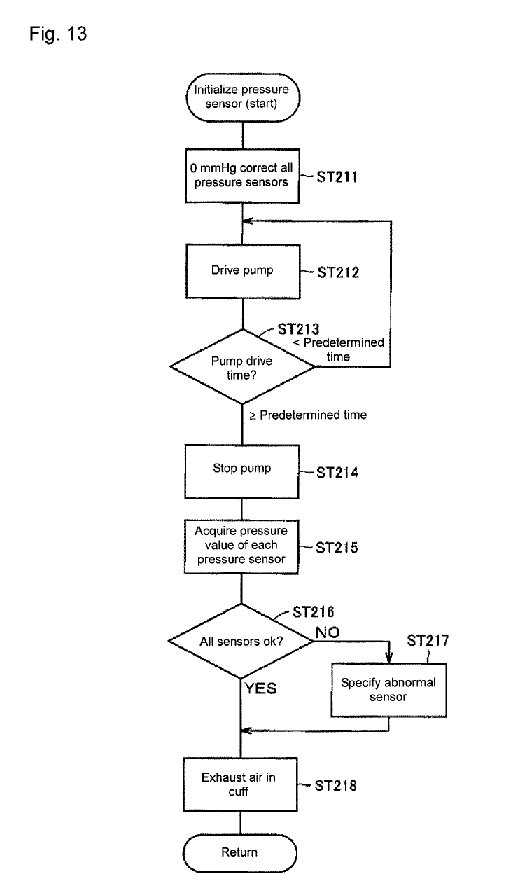

FIG. 13 shows the processing procedure of detecting sensor abnormality in the initialization of the pressure sensors in the electronic sphygmomanometer of the second embodiment. The flowchart in FIG. 13 is performed in step ST3 described above. In the second embodiment, the processes other than the process in step ST3 are similar to those in the processing procedure of the blood pressure measurement in the first embodiment, and thus the description thereof will not be repeated.

In the second embodiment, the processes in steps ST1 to ST3 described in FIG. 4 are executed in the non-usage state (the state where the cuff 150A (150B) is attached to the main body 110A (110B)). Thereafter, the person to be measured attaches the cuff 150A (150B) to the upper arm, so that the electronic sphygmomanometer transitions from the non-usage state to the usage state. The blood pressure measurement processes from step ST4 are performed in the usage state.

With reference to FIG. 13, the 0 mmHg correction of all the pressure sensors is performed in the initialization of the pressure sensors in the non-usage state (step ST211).

Thereafter, the pump 51 is driven for a constant period of time at a predetermined voltage by the pump drive circuit 53 to feed a predetermined amount of air into the air bag 21 (steps ST212 to ST214). The pressure sensor abnormality detection unit 113 detects the output values of the first and second pressure sensors 321 and 322 (step ST215). A difference between the detected output values is then calculated, and the calculated difference and a predetermined value are compared with each other to detect the abnormality of the pressure sensor based on the comparison result (step ST216).

Specifically, determination is made that all the pressure sensors are normal (YES in step ST216) if detected that the difference is smaller than or equal to the predetermined value based on the comparison result. To the contrary, if detected that the difference has a value greater than the predetermined value (NO in step ST216), the abnormal sensor is specified (step ST217). The method described above (abnormality detection of sensor) described above can be used to this specification of the abnormal sensor. Information indicating the specified abnormal pressure sensor is displayed on the display unit 40.

Thereafter, the air in the air bag 21 of the cuff 20 is exhausted (step ST218). The initialization of the pressure sensors (step ST3) is thereby terminated.

### Third Embodiment

The configuration for detecting the abnormality of the pressure sensor is not limited to those described in the first and second embodiments, but may be detected by the configuration according to a third embodiment.

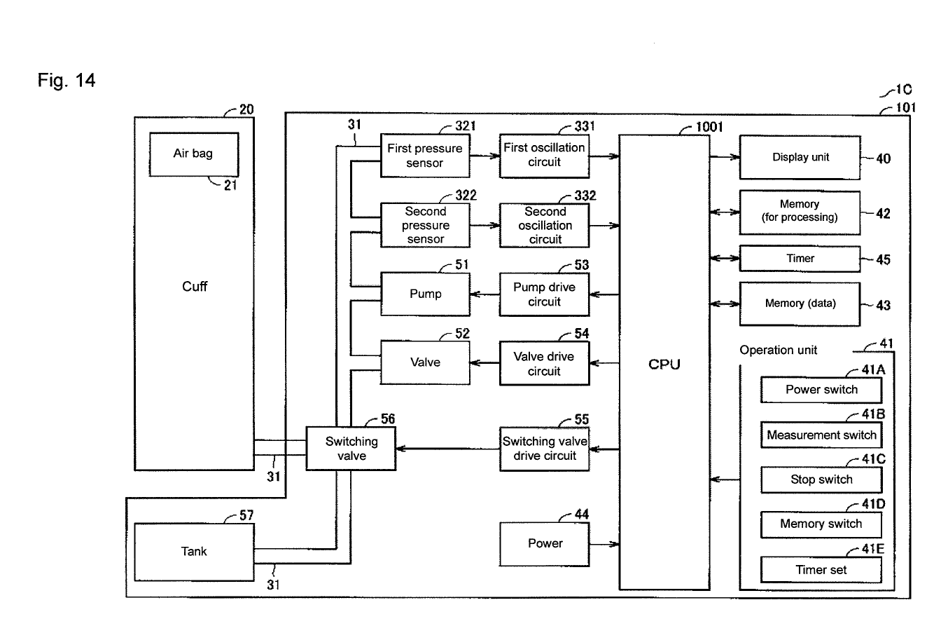

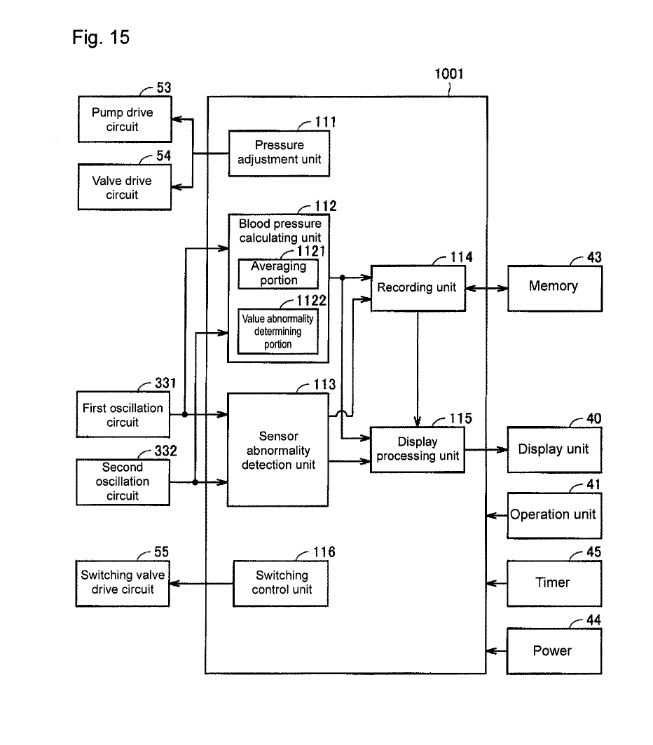

FIG. 14 and FIG. 15 show a hardware configuration and a function configuration of an electronic sphygmomanometer 1C according to the third embodiment.

Comparing the configuration of FIG. 14 and the configuration of FIG. 2 according to the first embodiment, the difference therebetween is that the electronic sphygmomanometer 1C in FIG. 14 includes a main body 101 in place of the main body 10 in FIG. 2.

In addition to the configuration of FIG. 2, the main body 101 includes therein a tank 57 for storing a constant volume of air, a switching valve 56 connected to the tank 57 through the air tube 31, and a switching valve drive circuit 55 for controlling the opening and closing operations of the switching valve 56. The main body 101 in FIG. 14 includes a CPU 1001 in place of the CPU 100 in FIG. 2.

The air tube 31 is connected to the switching valve 56. The air tube 31 includes an air tube (hereinafter referred to as first air tube) commonly connected to the first and second pressure sensors 321 and 322, the pump 51, and the valve 52, an air tube (hereinafter referred to as second air tube) connected to the cuff 20 (air bag 21), and an air tube (hereinafter referred to as third air tube) connected to the tank 57.

Comparing the configuration of FIG. 15 and the configuration of FIG. 3 according to the first embodiment, the difference therebetween is that the electronic sphygmomanometer 1C in FIG. 15 includes the CPU 1001 in place of the CPU 100 in FIG. 3. With reference to FIG. 15, the CPU 1001 includes a switching control unit 116 in addition to the configuration of FIG. 3. The switching control unit 116 controls the switching valve drive circuit 55.

### (Another Example of Abnormality Detection in Initialization of Pressure Sensor)

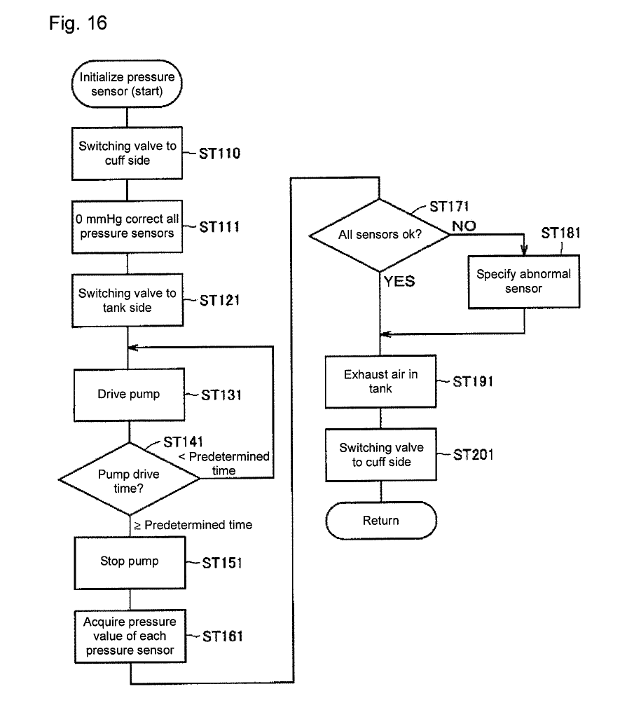

FIG. 16 shows the processing procedure of detecting sensor abnormality in the initialization of the pressure sensors. The flowchart in FIG. 16 is performed in step ST3 described above. In the third embodiment, the processes other than the process in step ST3 are similar to those in the processing procedure of the blood pressure measurement according to the first embodiment, and thus the description thereof will not be repeated.

In the abnormality detection process according to the third embodiment, the output values of the first and second pressure sensors 321 and 322 in a case where a predetermined amount of air is fed to the tank 57 are compared with each other. If detected that the difference between the output values exceeds a predetermined value (e.g., 5 mmHg) based on the comparison result, one of the pressure sensors is determined as abnormal.

Assume that the switching valve 56 is switched to the tank 57 side prior to the initialization of the pressure sensors. With reference to FIG. 16, the switching control unit 116 first outputs a control signal to the switching vale drive circuit 55 in the initialization of the pressure sensors. The switching valve drive circuit 55 switches the switching valve 56 from the tank 57 side to the cuff 20 side based on the control signal (step ST110). Therefore, the first air tube and the second air tube are connected through the switching valve 56, and the flow path of the air is configured by both of the tubes. The 0 mmHg correction of all the pressure sensors is carried out in such a state (step ST111).

Thereafter, the switching control unit 116 outputs a control signal to the switching valve drive circuit 55. The switching valve drive circuit 55 switches the switching valve 56 from the cuff 20 side to the tank 57 side based on the control signal. Therefore, the first air tube is disconnected from the second air tube and is connected to the third air tube through the switching valve 56. The air flows to the tank 57 by the switching valve 56 (step ST121).

Thereafter, the pump 51 is driven for a constant period of time at a predetermined voltage by the pump drive circuit 53 to feed a predetermined amount of air into the tank 57 (steps ST131 to ST151). The pressure sensor abnormality detection unit 113 detects the output values of the first and second pressure sensors 321 and 322 at this time (step ST161). The difference between the detected output values is then calculated, and the calculated difference and a predetermined value are compared with each other to detect the abnormality of the pressure sensor based on the comparison result (step ST171).

Specifically, determination is made that all the pressure sensors are normal (YES in step ST171) if detected that the difference is smaller than or equal to the predetermined value based on the comparison result. To the contrary, if detected that the difference has a value greater than the predetermined value (NO in step ST171), the abnormal sensor is specified (step ST181). The method (abnormality detection of sensor) described above can be used to specify the abnormal sensor. Information indicating the specified abnormal pressure sensor is displayed on the display unit 40.

The air in the tank 57 is thereafter exhausted (ST191). The switching control unit 116 then outputs a control signal to the switching valve drive circuit 55. The switching valve drive circuit 55 switches the switching valve 56 from the tank 57 side to the cuff 20 side based on the control signal. Therefore, the first air tube is disconnected from the third air tube and is connected to the second air tube through the switching valve 56. The air flows to the cuff 20 by the switching valve 56 (step ST201). The initialization of the pressure sensors (step ST3) is thereby terminated.

### (Display Example)

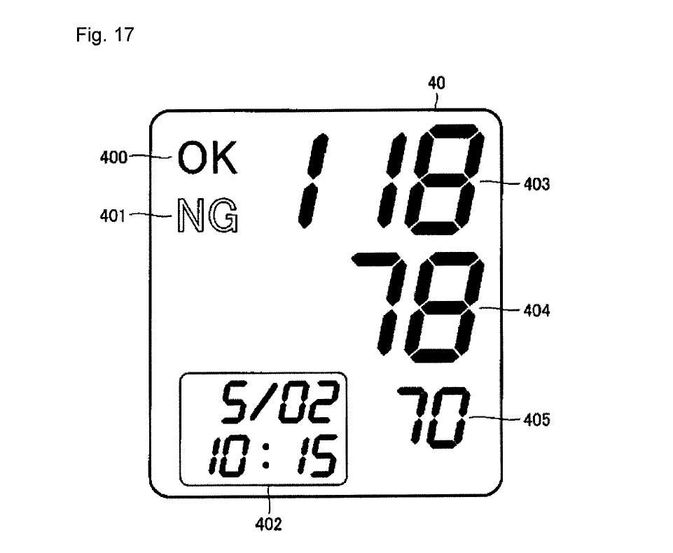

FIG. 17 to FIG. 19 each show a display example of the abnormality detection result of the pressure sensor on the display unit 40.

In FIG. 17, the display processing unit 115 does not light characters “NG” and lights only characters “OK” if at least one of the first or second pressure sensors 321 or 322 is normal. If all the sensors are abnormal, the characters “OK” are not lighted and the characters “NG” are lighted. In FIG. 17, there are simultaneously displayed data 402 of the measurement time measured by the timer 45, data 403 of the systolic blood pressure, data 404 of the diastolic blood pressure, and data 405 of the pulse rate data as a result of the blood pressure measurement, as well as the display of “NG”/“OK”.

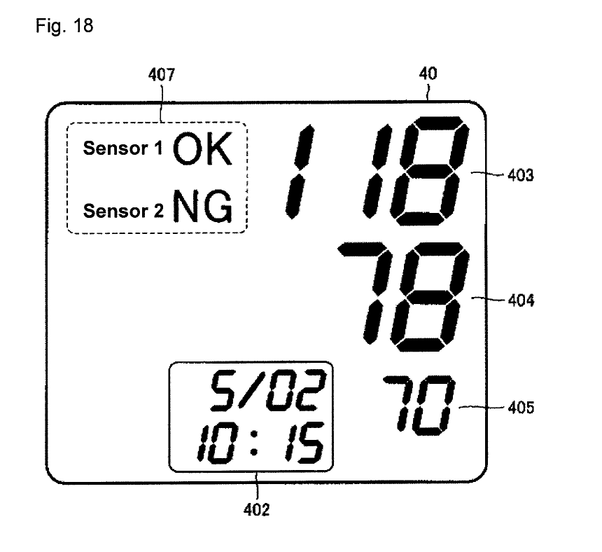

FIG. 18 shows an example of displaying “NG”/“OK” for each of the pressure sensors. In this case, the display of “NG”/“OK” is made in a message 407 for each of the pressure sensors. According to FIG. 18, it is found that the first pressure sensor 321 is normal but the second pressure sensor 322 is abnormal.

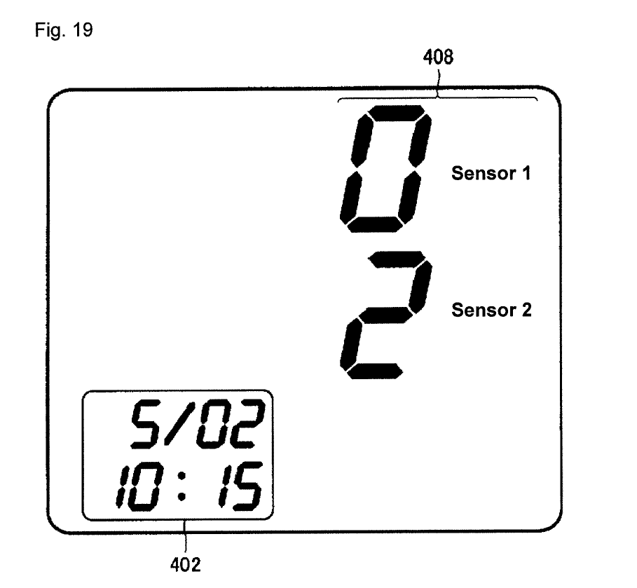

FIG. 19 shows an example in which the current detected pressure value is displayed for each of the pressure sensors in the standby state or the like where the electronic sphygmomanometer is not used for the blood pressure measurement. In this case, there is indicated that the current pressure value detected by the first pressure sensor 321 is 0 mmHg and the current pressure value detected by the second pressure sensor 322 is 2 mmHg by a message 408.

The person to be measured can obtain a timing of requesting the manufacturer for the calibration of the pressure sensors by checking the displays shown in FIG. 17 to FIG. 19. Therefore, the blood pressure measurement can be avoided from being carried out without noticing the abnormality in the pressure sensor, and the reliability of the blood pressure measurement value can be enhanced.

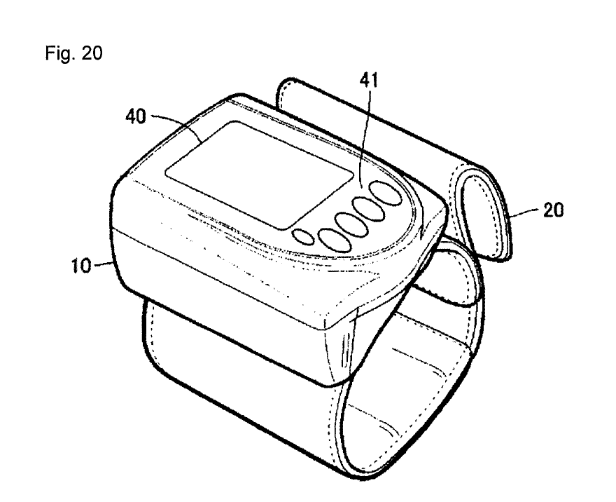

In the above embodiments, the electronic sphygmomanometer is of a mounting type and the cuff 20 is wrapped around the upper arm, but the function and the configuration of the abnormality detection of the pressure sensor described in the embodiments can be similarly applied to a wrist type electronic sphygmomanometer in which the cuff 20 and the main body 10 are integrally configured and the cuff 20 is to be wrapped around the wrist as shown in FIG. 20.

### (Timing of Abnormality Detection by Sensor Detection Unit 113)

The sensor abnormality detection unit 113 may carry out the detection operation when the measurement data is read out from the memory 43 and the read measurement data is displayed on the display unit 40 in response to the operation of the memory switch 41D. Alternatively, the detection operation may be carried out when the time of the timer 45 is adjusted.

In a case where the power supply 4 is provided with a battery, the sensor abnormality detection unit 113 may carry out the abnormality detection of the pressure sensor immediately after the battery of the power supply 44 is changed to a new battery. Alternatively, in a case where the power is supplied to the power supply 44 from an external power supply (commercial power supply, etc.) through an AC (Alternating Current) adapter, the sensor abnormality detection unit 113 may carry out the abnormality detection of the pressure sensor immediately after the supply of power from the external power supply to the power supply 44 is started.

Further alternatively, the sensor abnormality detection unit 113 may carry out the detection operation in response to an instruction to be input from outside through the operation unit 41.

### (Timing of Display of Sensor Abnormality Detection Result)

The result detected by the sensor abnormality detection unit 113 may be displayed in the standby state before the start of the blood pressure measurement, or may be displayed when displaying the blood pressure measurement result in step ST11.

Alternatively, the result of the abnormality detection may be displayed every time the detection is carried out by the sensor abnormality detection unit 113. The most recent abnormality detection result may be read out from the memory 43 to be displayed in response to an instruction input from outside through the operation unit 41.

The embodiments disclosed herein are illustrative in all aspects and should not be construed as being restrictive. While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

The present invention is useful, for example, in a device for measuring a blood pressure using a pressure sensor.

### DESCRIPTION OF SYMBOLS

1, 1A, 1B, 1C electronic sphygmomanometer

55 switching valve drive circuit

56 switching valve

57 tank

321 first pressure sensor

322 second pressure sensor

331 first oscillation circuit

332 second oscillation circuit

112 blood pressure calculating unit

113 sensor abnormality detection unit

1. An electronic sphygmomanometer comprising:
a cuff to be attached to a measurement site;
a pressurization/depressurization unit for adjusting a pressure to be applied to the cuff;
a pressure detection unit including a plurality of pressure sensors, for detecting cuff pressures in the cuff based on pressure information output from the plurality of pressure sensors; and
a blood pressure calculating unit for calculating a blood pressure based on a change in the cuff pressures detected by the pressure detection unit,
wherein blood pressure measurement and detection of abnormality on the plurality of pressure sensors are carried out based on the cuff pressures respectively corresponding to the plurality of pressure sensors detected according to the pieces of pressure information output from the plurality of pressure sensors.

2. The electronic sphygmomanometer according to claim 1, wherein the blood pressure calculating unit calculates the blood pressure based on the cuff pressures respectively corresponding to the plurality of pressure sensors.

3. The electronic sphygmomanometer according to claim 1, further comprising:
an abnormality detecting portion for detecting abnormality of the plurality of pressure sensors,
wherein the abnormality detecting portion mutually compares the pieces of pressure information respectively corresponding to the plurality of pressure sensors and detects that at least one of the plurality of pressure sensors is abnormal based on a comparison result.

4. The electronic sphygmomanometer according to claim 3, wherein the blood pressure calculating unit excludes the cuff pressure corresponding to the pressure sensor that is detected as being abnormal, out of the plurality of pressure sensors from data used to calculate the blood pressure.

5. The electronic sphygmomanometer according to claim 3, further comprising:
a storage unit for storing data of the blood pressure calculated by the blood pressure calculating unit,
wherein a detection result by the abnormality detecting portion is stored in the storage unit in association with the data of the blood pressure.

6. The electronic sphygmomanometer according to claim 5, wherein the pressure information is detected in a process of increasing or in a process of decreasing the pressure to be applied to the cuff by the pressurization/depressurization unit.

7. The electronic sphygmomanometer according to claim 3, wherein the pressure information is detected in a process of increasing or in a process of decreasing the pressure to be applied to the cuff by the pressurization/depressurization unit.

8. The electronic sphygmomanometer according to claim 3, wherein
the abnormality detecting portion mutually compares the pieces of pressure information respectively corresponding to the plurality of pressure sensors at a predetermined cuff pressure, and detects that at least one of the plurality of pressure sensors is abnormal based on a comparison result, and
the predetermined cuff pressure corresponding to each of the plurality of pressure sensors indicates a difference between pressure information output by the pressure sensor when a pressure of 0 mmHg is applied to the cuff in measuring the blood pressure and pressure information output by the pressure sensor when the pressure of 0 mmHg is applied to the cuff and acquired preliminarily in calibration of the pressure sensor.

9. The electronic sphygmomanometer according to claim 3, wherein the pieces of pressure information respectively corresponding to the plurality of pressure sensors are detected in a state where the cuff is wrapped around a member in a circular column shape and a predetermined amount of air is flowed into the cuff

10. The electronic sphygmomanometer according to claim 9, further comprising:
a main body separate from the cuff, wherein
the main body includes the pressurization/depressurization unit, the pressure detection unit, the blood pressure calculating unit, and the abnormality detecting portion, and
a housing of the main body is configured by member in the circular column shape.

11. The electronic sphygmomanometer according to claim 3, further comprising:
a tank for storing a predetermined amount of air;
a first air flow path communicating to the pressurization/depressurization unit and to the pressure detection unit;
a second air flow path communicating to the cuff;
a third air flow path communicating to the tank; and
a flow path switching unit for selectively connecting one of the second air flow path and the third air flow path to the first air flow path,
wherein when the predetermined amount of air is flowed into the tank after the flow path switching unit connects the third air flow path to the first air flow path, the pressure detection unit detects the cuff pressure in the cuff based on the pieces of pressure information output from the plurality of pressure sensors.
